(19) United States
(12) Patent Application Publication
(10) Pub. No.: US 2026/0265658 A1
(43) Pub. Date: Sep. 10, 2026

(54) BIOLOGICAL FERMENTATION REACTOR BASED ON ROTARY JET STIRRING AND METHOD THEREOF

(71) Applicant: NANJING TECH UNIVERSITY, Nanjing (CN)

(72) Inventors: Hong XU, Nanjing (CN); Rui WANG, Nanjing (CN); Qian CHEN, Nanjing (CN); Quanfei LI, Nanjing (CN); Peng LEI, Nanjing (CN); Sha LI, Nanjing (CN); Yian GU, Nanjing (CN); Liang SUN, Nanjing (CN)

(73) Assignee: NANJING TECH UNIVERSITY, Nanjing (CN)

(21) Appl. No.: 19/163,881

(22) PCT Filed: May 13, 2024
(86) PCT No.: PCT/CN2024/092765

§ 371 Date: Sep. 10, 2025

(30) Foreign Application Priority Data
May 15, 2023 (CN) 202310546049.6

(51) Int. Cl. C12M 1/04; C12M 1/00; C12M 1/02; C12M 1/12; C12M 1/34

(52) U.S. Cl. CPC C12M 27/04; C12M 29/14; C12M 37/02; C12M 37/04; C12M 41/22; C12M 41/26; C12M 41/34

(57) ABSTRACT

It discloses a biological fermentation reactor based on rotary jet stirring and method thereof. The reactor comprises a fermentation tank, a feeding aeration circulation system and a high-temperature steam sterilization device; the fermentation tank is connected with the feeding aeration circulation system through an internal circulation pipeline, one end of which is connected with a bottom of the fermentation tank, and the other end is connected with a rotary jet stirring device located in the fermentation tank, and the internal circulation pipeline is provided with a vacuum pump; the feeding aeration circulation system is connected onto the internal circulation pipeline, and is used for providing a material and a gas source needed for fermentation into the pipeline; and the high-temperature steam sterilization device is connected with the feeding aeration circulation system and used for providing high-temperature steam to a whole system and the fermentation tank for sterilization.

### CROSS-REFERENCE TO RELATED APPLICATIONS

This application is the U.S. national phase of International Application No. PCT/CN2024/092765 filed on 13 May 2024 which designated the U.S. and claims priority to Chinese Application No. CN 202310546049.6 filed on 15 May 2023, the entire contents of each of which are hereby incorporated by reference.

### TECHNICAL FIELD

The present invention belongs to the field of biological fermentation, in particular to a biological fermentation reactor based on rotary jet stirring and a method.

### BACKGROUND

At present, the era of bio-economy marked by bio-manufacturing industry is coming, and all major developed economies in the world have identified biotechnology as one of the key technologies for economic and technological development in the 21^{st} century. However, as a foundation of bio-manufacturing, fermentation industry still has great limitations, especially mass transfer optimization of a fermentation system, which is the most important factor limiting an output scale. How to improve a transfer efficiency of gas in a fermentation broth has become the focus of the industry.

In aerobic fermentation, high oxygen supply and low utilization rate are the main factors that limit the output thereof, which meanwhile, cause great waste of resources. In order to solve this problem, the following methods have been put forward: (1) A type and a combination of stirring paddles are changed to optimize a stirring mode to promote full mixing of fluids. Although this method has a better mixing effect than conventional stirring, due to the increase of a number and arrangement of paddles, a power of a driving motor is increased, which leads to serious local heating of a stirring rod, inhibiting vitality of microorganisms to a certain extent and causing a waste of electricity. (2) An oxygen carrier is added, which is an organic phase that is immiscible with water, non-toxic to microorganisms, and has high oxygen solubility. Because a system formed by the oxygen carrier and the fermentation broth has the characteristics of high oxygen transfer speed, low energy consumption, less bubble formation and small shearing force, the oxygen carrier has attracted more and more attention in the field of biochemical engineering. However, specific oxygen carriers need to be screened for different fermentation systems, so this method is not universal. 3) By means of gene engineering, a recombinant such as vitreoscilla hemoglobin (VHb) which can promote oxygen absorption and utilization was transformed into a fermentation strain by means of gene engineering, so as to improve an oxygen uptake capacity of the bacteria. This technology is currently in a laboratory stage and has a prospect of large-scale production.

To sum up, how to use a simple method to improve a mass transfer effect without increasing energy consumption and reducing influences on the bacteria, so as to promote a metabolic ability of the strains to meet the demand of increasing production has become an urgent technical problem in the industry.

### SUMMARY

Object of the present invention: a technical problem to be solved by the present invention is to provide a biological fermentation reactor with enhanced mass transfer, aiming at the problem of insufficient mass transfer effect in the microbial fermentation process in the industry, so as to improve full mixing of fluids in a tank, provide a good environment for growth, metabolism and production of various bacteria, and promote yield increase.

In order to achieve the above-mentioned object, the present invention employs the following technical solutions.

A biological fermentation reactor based on rotary jet stirring comprises a fermentation tank, a feeding aeration circulation system and a high-temperature steam sterilization device; the fermentation tank is connected with the feeding aeration circulation system through an internal circulation pipeline, one end of which is connected with a bottom of the fermentation tank, and the other end is connected with a rotary jet stirring device located in the fermentation tank, and the internal circulation pipeline is provided with a vacuum pump; the feeding aeration circulation system is connected onto the internal circulation pipeline, and is used for providing a material and a gas source needed for fermentation into the pipeline; and the high-temperature steam sterilization device is connected with the feeding aeration circulation system and used for providing high-temperature steam to a whole system and the fermentation tank for sterilization.

Specifically, the rotary jet stirring device comprises a rotary paddle sprayer and a hollow pipe shaft; the rotary paddle sprayer is rotatably installed at a bottom of the hollow pipe shaft, and the hollow pipe shaft is longitudinally penetrated and installed at a top of the fermentation tank and connected with the internal circulation pipeline; and the rotary paddle sprayer has a hollow structure, and the material and the gas from the internal circulation pipeline form jet injection from the rotary paddle sprayer and provide power for rotation of the rotary paddle sprayer.

Specifically, the feeding aeration circulation system comprises a first branch pipe and a second branch pipe which are connected in parallel on the internal circulation pipeline in sequence; the first branch pipe is provided with a feeding groove for supplying a fermentation material or a cleaning liquid to the internal circulation pipeline, and a front end of the first branch pipe is connected to the empty water pump on the internal circulation pipeline; and the second branch pipe is sequentially provided with an air compressor and a sterilizer for providing a fermentation gas to the internal circulation pipeline, and a front end of the second branch pipe is connected to the internal circulation pipeline through an aeration module.

Further, the high-temperature steam sterilization device is respectively connected to the feeding groove and the second branch pipe at a front end of the air compressor through the two branch pipes, and sends high-temperature steam to the first branch pipe and the second branch pipe respectively; and
a first valve is arranged one end of the internal circulation pipeline close to the bottom of the fermentation tank; a second valve is arranged between the empty water pump and the aeration module on the internal circulation pipeline; a third valve is arranged between the feeding groove and the empty water pump on the internal circulation pipeline; a fourth valve is arranged between the air compressor and the sterilizer on the second branch pipe; and a fifth valve is arranged on a pipeline connecting the high-temperature steam sterilization device and the second branch pipe.

Specifically, the fermentation tank comprises a tank body and a tank cap; the tank cap is sealed on a top of the tank body, and the tank cap is provided with an exhaust pipe; an outer wall of the tank body is provided with a heating jacket, and a temperature probe, a pH probe and a dissolved oxygen probe are installed on the outer wall; a bottom of the tank body is provided with a sampling port and a discharging port; the tank cap is further provided with a cooler; and the cooler is a U-shaped pipe, which is inserted on the tank cap and is provided with a water inlet and a water outlet, a serpentine surrounding design is employed on a part of the cooler located in the tank body, and a material in the tank body is cooled quickly by cooling water.

Further, the aeration module is made of a sintered porous alloy material, with a pore size ranging from 5 μm to 5 nm. A microporous material of a specific shape formed by metal powder such as stainless steel sintered at high temperature is usually selected for the aeration module.

Further, the sterilizer is composed of multiple layers of polymer material and ceramic filter membranes with pore sizes less than 0.45 μm and 0.22 μm in series and encapsulated by a stainless steel jacket.

Further, a top of the feeding groove is provided with a sterile exhaust port, internally provided with a stirring and blending device is built in for pre-mixing preparation of a culture medium, and is equipped with a sealing gland to always keep a closed and sterile state during fermentation.

Further, the temperature probe, the pH probe and the dissolved oxygen probe are respectively arranged in a circle at an equal distance of 120 degrees in the tank body, and are arranged in three circles from top to bottom.

Furthermore, the present application also provides a method for performing biological aerobic or anaerobic fermentation by using the biological fermentation reactor above, comprising the following steps:
(1) sealing the fermentation tank completely, and performing high-temperature steam sterilization on the fermentation tank and the feeding aeration circulation system by the high-temperature steam sterilization device;
(2) inoculating a seed solution into the feeding aeration circulation system by aseptic inoculation, and then sending the seed solution into the fermentation tank through the internal circulation pipeline;
(3) continuously conveying a culture medium and a fermentation gas into the fermentation tank through the feeding aeration circulation system, and meanwhile, internally circulating materials in the fermentation tank through the internal circulation pipeline and the rotary jet stirring device, and enhancing mass transfer and efficiency; and
(4) after the fermentation is completed, collecting a fermentation broth, feeding a cleaning liquid into the fermentation tank through the feeding aeration circulation system, and completing cleaning of the whole set of device, and finally adopting the high-temperature steam sterilization device for sterilization.

Beneficial effects:
(1) Different from a conventional stirring paddle for blending, the rotary jet stirring sprayer adopted in the present invention can be used for three-dimensional jet stirring by driving the sprayer with circulating fermentation broth, thus solving the problems of uneven heat dissipation and power consumption caused by motor stirring, effectively improving a dead zone problem of the conventional stirring paddle and promoting the full mixing of the fluid in the tank.
(2) The present invention uses aeration and jet to replace ordinary built-in gas supply operation, so that gas enters the reaction system in smaller size, and a micron-sized gas source is mixed in a process of circulating a fermentation broth, thus effectively improving dissolution efficiency of gas and promoting metabolism of bacteria.
(3) Due to the comprehensive design of the present invention, the fermentation medium does not need to be externally configured in advance and placed in a sterilization pot for sterilization, and only needs to be poured into the feeding groove according to the formula, and the system can automatically sterilize and pump the medium into the fermentation tank, thus simplifying tedious preparation work.
(4) According to the present invention, the fermentation tank with self-cleaning integrated design does not need to be disassembled and cleaned, so that a labor cost is reduced, and a use stability of the device is ensured. An ability of rapid cleaning can be improved, a production time can be effectively improved, use of water and cleaning agents are reduced, and a cleaning cost is decreased. Strict requirements of aerobic, anaerobic and other gas fermentation can be satisfied. Different from the traditional post-fermentation cleaning, the biological fermentation reactor of the present invention can save a lot of water resources and labor costs by adopting a self-cleaning method. The design of automatic mixing and medium feeding can achieve the effect of efficient mixing and simple feeding, which is helpful to simplify the complicated pretreatment work before fermentation.

### BRIEF DESCRIPTION OF THE DRAWINGS

The advantages of the above and/or other aspects of the present invention will become more apparent by further explaining the present invention with reference to the following drawings and detailed description.

FIG. 1 is a schematic diagram showing an overall structure of a biological fermentation reactor according to the present invention.

Reference numerals respectively represent: 1—fermentation tank; 2—feeding aeration circulation system; 3—high-temperature steam sterilization device; 4—tank body; 5—tank cap; 6—temperature probe; 7—pH probe; 8—dissolved oxygen probe; 9—cooler; 10—rotary jet stirring device; 11—sampling port; 12—discharging port; 13—exhaust pipe; 14—heating jacket; 15—vacuum pump; 16—feeding groove; 17—aeration module; 18—sterilizer; 19—air compressor; 20—first valve; 21—second valve; 22—third valve; and 23—fourth valve.

FIG. 2 is a feeding groove provided with a stirring and blending device in the biological fermentation reactor according to the present invention. 24—stirring paddle; and 27—sterile exhaust port.

FIG. 3 is a sterilizer in the biological fermentation reactor according to the present invention. 25—series filter membrane for sterilization, and 26—stainless steel jacket.

FIG. 4 shows yield and biomass results of polyglutamic acid produced by using the biological fermentation reactor of the present invention by aerobic fermentation.

FIG. 5 shows yield results of polyglutamic acid produced by using a common mechanically stirred biological fermentation tank by aerobic fermentation.

FIG. 6 is a comparison diagram of yields of polyglutamic acid produced by using the reactor of the present invention and the common device by aerobic fermentation.

FIG. 7 is a comparison diagram of biomasses of polyglutamic acid produced by using the reactor of the present invention and the common device by aerobic fermentation.

### DETAILED DESCRIPTION

The present invention can be better understood from the following embodiments.

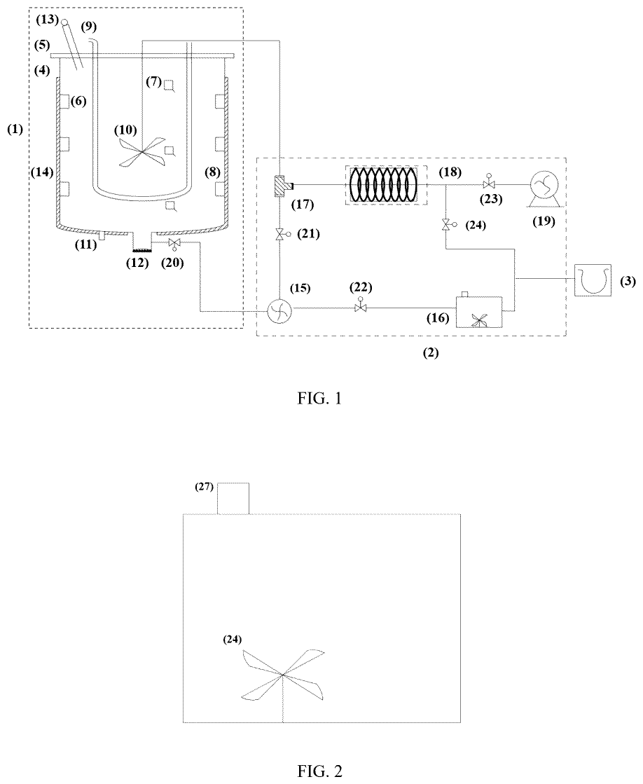

As shown in FIG. 1, a biological fermentation reactor according to the present invention comprises a fermentation tank 1, a feeding aeration circulation system 2 and a high-temperature steam sterilization device 3. The fermentation tank 1 is connected with the feeding aeration circulation system 2 through an internal circulation pipeline, one end of the internal circulation pipeline is connected with a bottom of the fermentation tank, the other end of the internal circulation pipeline is connected with a rotary jet stirring device 10 located in the fermentation tank, and the internal circulation pipeline is provided with a vacuum pump 15. The feeding aeration circulation system 2 is connected onto the internal circulation pipeline, and is used for providing a material and a gas source needed for fermentation into the pipeline. The high-temperature steam sterilization device 3 is connected with the feeding aeration circulation system 2 and used for providing high-temperature steam to a whole system and the fermentation tank for sterilization.

The rotary jet stirring device 10 comprises a rotary paddle sprayer and a hollow pipe shaft. The rotary paddle sprayer is rotatably installed at a bottom of the hollow pipe shaft, and the hollow pipe shaft is longitudinally penetrated and installed at a top of the fermentation tank and connected with the internal circulation pipeline. The rotary paddle sprayer has a hollow structure, and the material and the gas from the internal circulation pipeline form jet injection from the rotary paddle sprayer and provide power for rotation of the rotary paddle sprayer. Based on a jet rotation technology and the aeration device, the present invention refines sterile air in a flowing process of a fermentation broth, promotes a gas dissolving effect, comprising but not limited to oxygen, nitrogen, carbon dioxide, or the like, and is suitable for the fermentation of various aerobic and anaerobic special gases.

The feeding aeration circulation system 2 comprises a first branch pipe and a second branch pipe which are connected in parallel on the internal circulation pipeline in sequence. The first branch pipe is provided with a feeding groove 16 for supplying a fermentation material or a cleaning liquid to the internal circulation pipeline, and a front end of the first branch pipe is connected to the empty water pump 15 on the internal circulation pipeline. The second branch pipe is sequentially provided with an air compressor 19 and a sterilizer 18 for providing a fermentation gas to the internal circulation pipeline, and a front end of the second branch pipe is connected to the internal circulation pipeline through an aeration module 17.

The high-temperature steam sterilization device 3 is respectively connected to the feeding groove 16 and the second branch pipe at a front end of the air compressor 19 through the two branch pipes, and sends high-temperature steam to the first branch pipe and the second branch pipe respectively.

A first valve 20 is arranged one end of the internal circulation pipeline close to the bottom of the fermentation tank 1. A second valve 21 is arranged between the empty water pump 15 and the aeration module 17 on the internal circulation pipeline. A third valve 22 is arranged between the feeding groove 16 and the empty water pump 15 on the internal circulation pipeline. A fourth valve 23 is arranged between the air compressor 19 and the sterilizer 18 on the second branch pipe. A fifth valve 24 is arranged on a pipeline connecting the high-temperature steam sterilization device 3 and the second branch pipe.

Through the innovation of design, the present invention can improve the full blending of the fluid in the tank, provide a good environment for the growth, metabolism and production of various bacteria, and promote the yield increase. Moreover, the integrated fermentation device is suitable for metabolic needs of various bacteria, which can not only promote the yield increase by improving the mass transfer effect, but also effectively wash fermentation residue in the tank body by using a self-cleaning program, thus greatly saving labor cost. In addition, the simple linkage mode can solve construction land limitation and reduce a maintenance cost, which is of great significance and practical value for promoting the development of microbial fermentation reactors.

The fermentation tank 1 comprises a tank body 4 and a tank cap 5. The tank cap 5 is sealed on a top of the tank body 4, and the tank cap 5 is provided with an exhaust pipe 13. An outer wall of the tank body 4 is provided with a heating jacket 14, and a temperature probe 6, a pH probe 7 and a dissolved oxygen probe 8 are installed on the outer wall. A bottom of the tank body 4 is provided with a sampling port 11 and a discharging port 12. The tank cap 5 is further provided with a cooler 9. The cooler 9 is a U-shaped pipe, which is inserted on the tank cap 5 and is provided with a water inlet and a water outlet, a serpentine surrounding design is employed on a part of the cooler 9 located in the tank body 4, and a material in the tank body is cooled quickly by cooling water.

As shown in FIG. 2, a top of the feeding groove 16 is provided with a sterile exhaust port, internally provided with a stirring and blending device is built in for pre-mixing preparation of a culture medium, and is equipped with a sealing gland to always keep a closed and sterile state during fermentation.

The aeration module 17 is made of a sintered porous alloy material, with a pore size ranging from 5 μm to 5 nm, and is arranged in a joint of the feeding aeration circulation system and the internal circulation pipeline. In this embodiment, the aeration module 17 is purchased from Nanjing Shineking Biotech Co., Ltd.

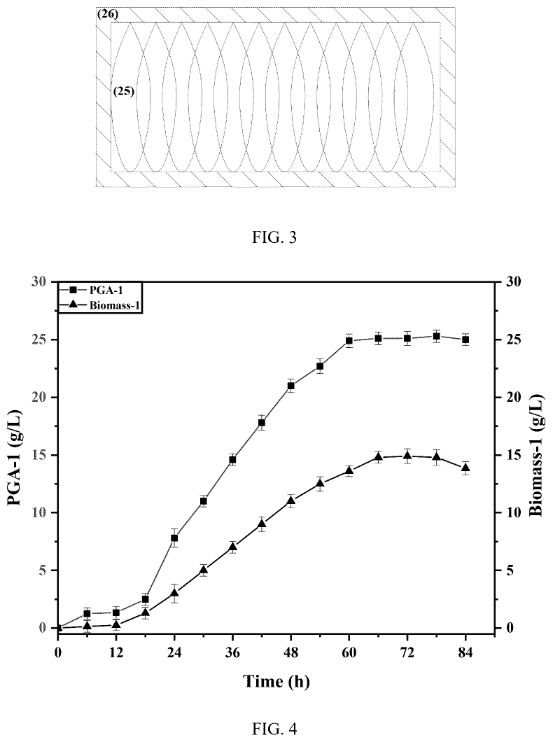

As shown in FIG. 3, the sterilizer 18 is composed of multiple layers of polymer material and ceramic filter membranes with pore sizes less than 0.45 μm and 0.22 μm in series and encapsulated by a stainless steel jacket 26. The polymer materials comprise but are not limited to PTFE, PES, PVDF, PA, or the like. In this embodiment, the sterilizer 18 is purchased from Nanjing Shineking Biotech Co., Ltd.

A body of the air compressor 19 comprises a switching valve which can be connected with various gas cylinders to meet living environment requirements of different fermentation strains. The high-temperature steam sterilization device is connected to the feeding aeration circulation system by a pipeline, and provides high-temperature steam for sterilization operation of the whole biological fermentation reactor.

The temperature probe 6, the pH probe 7 and the dissolved oxygen probe 8 on the tank body 4 are respectively arranged in a circle at an equal distance of 120 degrees in the tank body, and are arranged in three circles from top to bottom. There are nine probes, comprising three temperature probes 6, three pH probes 7 and three dissolved oxygen probes 8 respectively, and an average value of the three probes is taken as a reference value in use.

In the following examples, the following methods were used to prepare culture medium and activate strains, which were specifically as follows:
(1) Preparation of activated culture medium

A liquid culture medium Lysozyme Broth was used in this experiment, and the specific preparation process was as follows: firstly, 1-2 g of casein tryptone, 0.5-2.5 g of yeast extract and l-2 g of NaC1 powder are weighed and dissolved in 100 ml of distilled water, and then blended and sterilized in an autoclave at 121° C. for 30 min, and then stored in a refrigerator at 4° C. for later use.
(2) Activation of bacteria

Bacillus subtilis and Clostridium butyricum stored in a refrigerator at −80° C. were taken out and thawed, and three zones were scribed on a fixed plate respectively, and then inverted in an incubator at 37° C. for overnight culture. After the completion of the culture, it was confirmed by microscopic examination, and inoculating loops were used for inoculating one loop to 10 ml of small black flasks in LB liquid culture medium respectively for overnight culture and rejuvenation. After the rejuvenation, 1 ml of the bacteria solution was sucked into a pre-sterilized 100 ml LB broth conical flask (operating in a super clean bench), then the conical flask was placed on a constant temperature shaker (the temperature was set to be about 37° C.) and cultured for 12 h, the biomass was monitored in real time, and the solution was reserved when a logarithmic phase of growth was reached.

### Example 1

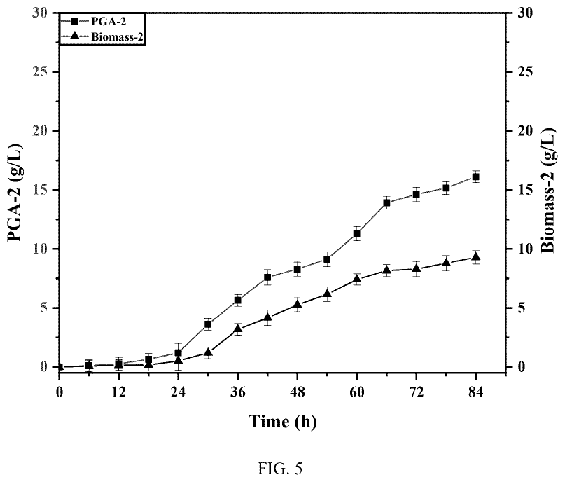

A biological fermentation reactor of the present invention was adopted for efficiently producing polyglutamic acid by aerobic fermentation.
(1) Formula of seed solution: 5 g/L of glucose, 5 g/L of beef extract, 5 g/L of peptone and 0.5 g/L of MgSO₄·7H₂O, wherein a pH was adjusted to 6.8 with NaOH solution. A solution flask was a 500 mL shake flask with 80-100 mL of seed solution.
(2) Formula of fermentation broth: 60-100 g/L of glucose, 50-60 g/L of sodium glutamate, 5-25 g/L of mora meal, 2 g/L of K₂HPO₄, and 0.1-5 g/L of MgSO₄, wherein a loaded broth was 60%-80% of a tank volume.
(3) First, a fermentation tank 1 was completely sealed, a first valve 20, a second valve 21 and a third valve 22 were opened, a fourth valve 23 was closed, a high-temperature steam sterilization device 3 was started to sterilize an whole system, then the first valve 20, the second valve 21 and the third valve 22 were closed, a culture medium was put into a feeding groove 16 as required, and built-in stirring was enabled and a high-temperature steam sterilization device 3 was started to sterilize the culture medium. After the sterilization, a seed solution of Bacillus subtilis was inoculated into the feeding groove 16 by aseptic inoculation, and pumped into the fermentation tank 1 together. Finally, the third valve 22 is closed, and the first valve 20, the second valve 21, the fourth valve 23 and the feeding aeration circulation system 2 were opened. In this case, an air compressor 19 was connected with an oxygen source or an air source, and meanwhile, all the devices in the fermentation tank 1 were put into operation, and the whole system entered a production stage of enhanced mass transfer and efficiency, during which it was sampled through a sampling port and a production curve was drawn as shown in FIG. 4. After the fermentation is completed, the first valve 20 was closed and a discharging port was opened to collect the fermentation broth, then the discharging port was closed, and the first valve 20, the second valve 21 and the third valve 22 were opened. A cleaning liquid was poured into the feeding groove 16, then a vacuum pump 15 was started, and a rotary jet stirring device 10 was driven by the cleaning liquid to carry out all-round cleaning on the tank body.

As shown in FIG. 4: with the start of the fermentation, at the initial stage of the fermentation (0-12 h), a biomass of Bacillus subtilis and a yield of polyglutamic acid were in a slow growth stage; at 12 h to 60 h, the Bacillus subtilis was in a logarithmic growth stage, and reached a maximum value of 14.9 g/L at 72 h, and then entered a phase of decline, and the yield of polyglutamic acid was positively correlated with the biomass of the Bacillus subtilis, with a highest yield reaching 25.3 g/L.

### Example 2

A biological fermentation reactor of the present invention was adopted for efficiently fermenting and producing microbial protein-Schizophyllum commune.
(1) Formula of seed solution: 25-30 g/L of water medium of potato glucose.
(2) Formula of fermentation broth: 55 g/L of glucose, 2.5 g/L of NaNO₃, 0.5 g/L of yeast cream, 1 g/L of KH₂PO₄, 0.5 g/L of MgSO₄·7H₂O, 1.5 g/L of citric acid, and 0.5 g/L of KCl.
(3) First, a fermentation tank 1 was completely sealed, a first valve 20, a second valve 21 and a third valve 22 were opened, a fourth valve 23 was closed, a high-temperature steam sterilization device 3 was started to sterilize an whole system, then the first valve 20, the second valve 21 and the third valve 22 were closed, a culture medium was put into a feeding groove 16 as required, and built-in stirring was enabled and a high-temperature steam sterilization device 3 was started to sterilize the culture medium. After the sterilization, Schizophyllum commune was inoculated into the feeding groove 16 by aseptic inoculation, and pumped into the fermentation tank 1 together. Finally, the third valve 22 is closed, and the first valve 20, the second valve 21, the fourth valve 23 and the feeding aeration circulation system 2 were opened. In this case, an air compressor 19 was connected with air, and meanwhile, all the devices in the fermentation tank 1 were put into operation, and the whole system entered a production stage of enhanced mass transfer and efficiency, during which it was sampled through a sampling port and a yield was measured. After the fermentation is completed, the first valve 20 was closed and a discharging port was opened to collect the fermentation broth, then the discharging port was closed, and the first valve 20, the second valve 21 and the third valve 22 were opened. A cleaning liquid was poured into the feeding groove 16, then a vacuum pump 15 was started, and a rotary jet stirring device 10 was driven by the cleaning liquid to carry out all-round cleaning on the tank body.

With the start of the fermentation, a content of Schizophyllum commune in the fermentation broth increased continuously. A number of Schizophyllum commune kept growing rapidly, and the final biomass could reach 30.45 g/L.

### Example 3

A biological fermentation reactor of the present invention was adopted for efficiently producing welan gum.
(1) Formula of seed solution (g/L): 20-50 of glucose, 0.1-5 of yeast powder, 2-4 of dipotassium phosphate, and 0.01-0.3 of magnesium sulfate at a pH of 6.86-7.4.
(2) Formula of fermentation broth (g/L): 20-50 of glucose, 2-4 of peptone, 2-4 of dipotassium phosphate and 0.01-0.3 of magnesium sulfate at a pH of 6.86-7.4, wherein a loaded broth was 60%-80% of a tank volume.
(3) First, a fermentation tank 1 was completely sealed, a first valve 20, a second valve 21 and a third valve 22 were opened, a fourth valve 23 was closed, a high-temperature steam sterilization device 3 was started to sterilize an whole system, then the first valve 20, the second valve 21 and the third valve 22 were closed, a culture medium was put into a feeding groove 16 as required, and built-in stirring was enabled and a high-temperature steam sterilization device 3 was started to sterilize the culture medium. After the sterilization, a seed solution of Bacillus subtilis was inoculated into the feeding groove 16 by aseptic inoculation, and pumped into the fermentation tank 1 together. (3) First, a fermentation tank 1 was completely sealed, a first valve 20, a second valve 21 and a third valve 22 were opened, a fourth valve 23 was closed, a high-temperature steam sterilization device 3 was started to sterilize an whole system, then the first valve 20, the second valve 21 and the third valve 22 were closed, a culture medium was put into a feeding groove 16 as required, and built-in stirring was enabled and a high-temperature steam sterilization device 3 was started to sterilize the culture medium. After the sterilization, alcaligenes NX-3 was inoculated into the feeding groove 16 by aseptic inoculation, and pumped into the fermentation tank 1 together. Finally, the third valve 22 is closed, and the first valve 20, the second valve 21, the fourth valve 23 and the feeding aeration circulation system 2 were opened. In this case, an air compressor 19 was connected with air, and meanwhile, all the devices in the fermentation tank 1 were put into operation, and the whole system entered a production stage of enhanced mass transfer and efficiency, during which it was sampled through a sampling port and a yield was measured. After the fermentation is completed, the first valve 20 was closed and a discharging port was opened to collect the fermentation broth, then the discharging port was closed, and the first valve 20, the second valve 21 and the third valve 22 were opened. A cleaning liquid was poured into the feeding groove 16, then a vacuum pump 15 was started, and a rotary jet stirring device 10 was driven by the cleaning liquid to carry out all-round cleaning on the tank body.

After measuring the yield of the welan gum, it was found that the yield of the strain reached 28 g/L after being cultivated in the fermentation system of jet enhanced stirring and mixing.

### Comparative Example 1

A common fermentation tank was used to produce polyglutamic acid by aerobic fermentation.
(1) Formula of seed solution: 20-30 g/L of glucose, 15-20 g/L of sodium glutamate, 5-15 g/L of yeast extract, 2-6 g/L of K₂HPO₄ and 0.1-0.5 of MgSO₄. A loaded solution was that a 500 mL shake flask was loaded with 80-100 mL of seed solution.
(2) Formula of fermentation broth: 60-100 g/L of glucose, 50-60 g/L of sodium glutamate, 5-25 g/L of (NH₄)SO₄, 2 g/L of K₂HPO₄, and 0.1-5 g/L of MgSO₄, wherein a loaded broth was 60%-80% of a tank volume.
(3) The culture medium above was placed in a fermentation tank, moved to a sterilization pot for sterilization, and then a controller was connected; after the temperature was cooled and stabilized, a seed solution of Bacillus subtilis was inoculated by a flame inoculation method, and sterile air or oxygen was introduced.

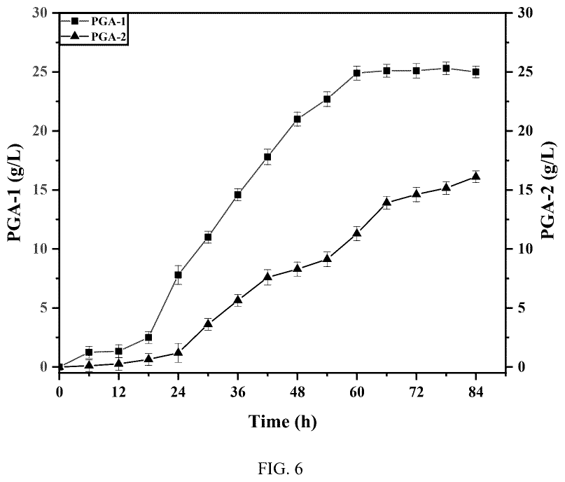

As shown in FIG. 5: with the start of the fermentation, at the initial stage of the fermentation (0-24 h), a biomass of Bacillus subtilis and a yield of polyglutamic acid were in a slow growth stage; at 24 h to 66 h, the Bacillus subtilis was in a logarithmic growth stage, and reached a maximum value of 9.3 g/L at 84 h, and then entered a phase of decline, and the yield of polyglutamic acid was positively correlated with the biomass of the Bacillus subtilis, with a highest yield reaching 16.12 g/L.

By comparing the yield of polyglutamic acid produced by aerobic fermentation between the reactor of the present invention and the common device in FIG. 6, it can be seen that the yield of polyglutamic acid is greatly increased by the reactor of the present invention, and compared with the common device, the yield of polyglutamic acid enters the vigorous production period at 18 h, the fermentation period is shortened by half, and the yield is increased by 56% compared with the common device.

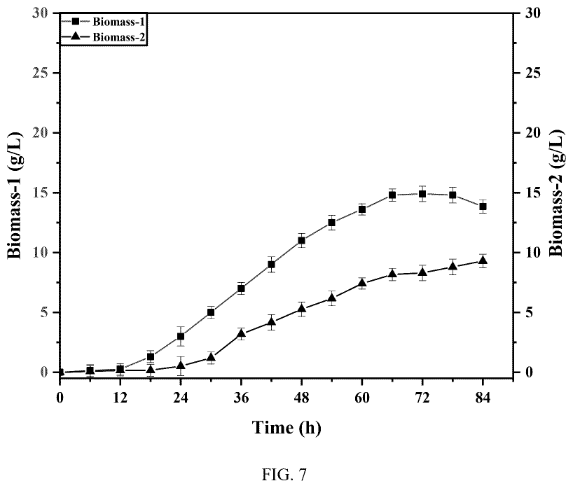

By comparing the biomass of polyglutamic acid produced by aerobic fermentation between the reactor of the present invention and the common device in FIG. 7, it can be seen that the growth of bacterial biomass is greatly promoted by the reactor of the present invention, which shows that the mixing of fluids and the improvement of mass transfer effect between gas and liquid are effectively promoted by introducing the aeration circulating fermentation broth to drive a rotary jet stirring sprayer.

To sum up, it further shows that the use of the biological fermentation reactor with enhanced mass transfer and self-cleaning can effectively shorten the fermentation period, improve the fermentation yield and improve the fermentation efficiency.

The present invention provides an idea and a method for the biological fermentation reactor based on rotary jet stirring and the method. There are many methods and ways to realize the technical solutions. The above are only the preferred embodiments of the present invention. It should be pointed out that those of ordinary skills in the art can make some improvements and embellishments without departing from the principle of the present invention, and these improvements and embellishments should also be regarded as falling with the scope of protection of the present invention. All the unspecified components in the embodiments can be realized by the prior art.

What is claimed is:

1. A biological fermentation reactor based on rotary jet stirring, comprising a fermentation tank (1), a feeding aeration circulation system (2) and a high-temperature steam sterilization device (3), wherein the fermentation tank (1) is connected with the feeding aeration circulation system (2) through an internal circulation pipeline, one end of the internal circulation pipeline is connected with a bottom of the fermentation tank (1), the other end of the internal circulation pipeline is connected with a rotary jet stirring device (10) located in the fermentation tank (1), and the internal circulation pipeline is provided with a vacuum pump (15); the feeding aeration circulation system (2) is connected onto the internal circulation pipeline, and is used for providing a material and a gas source needed for fermentation into the pipeline; and the high-temperature steam sterilization device (3) is connected with the feeding aeration circulation system (2) and used for providing high-temperature steam to a whole system and the fermentation tank for sterilization.

2. The biological fermentation reactor based on rotary jet stirring according to claim 1, wherein the rotary jet stirring device (10) comprises a rotary paddle sprayer and a hollow pipe shaft; the rotary paddle sprayer is rotatably installed at a bottom of the hollow pipe shaft, and the hollow pipe shaft is longitudinally penetrated and installed at a top of the fermentation tank (1) and connected with the internal circulation pipeline; and the rotary paddle sprayer has a hollow structure, and the material and the gas from the internal circulation pipeline form jet injection from the rotary paddle sprayer and provide power for rotation of the rotary paddle sprayer.

3. The biological fermentation reactor based on rotary jet stirring according to claim 2, wherein the feeding aeration circulation system (2) comprises a first branch pipe and a second branch pipe which are connected in parallel on the internal circulation pipeline in sequence; the first branch pipe is provided with a feeding groove (16) for supplying a fermentation material or a cleaning liquid to the internal circulation pipeline, and a front end of the first branch pipe is connected to the empty water pump (15) on the internal circulation pipeline; and the second branch pipe is sequentially provided with an air compressor (19) and a sterilizer (18) for providing a fermentation gas to the internal circulation pipeline, and a front end of the second branch pipe is connected to the internal circulation pipeline through an aeration module (17).

4. The biological fermentation reactor based on rotary jet stirring according to claim 3, wherein the high-temperature steam sterilization device (3) is respectively connected to the feeding groove (16) and the second branch pipe at a front end of the air compressor (19) through the two branch pipes, and sends high-temperature steam to the first branch pipe and the second branch pipe respectively; and
a first valve (20) is arranged one end of the internal circulation pipeline close to the bottom of the fermentation tank (1); a second valve (21) is arranged between the empty water pump (15) and the aeration module (17) on the internal circulation pipeline; a third valve (22) is arranged between the feeding groove (16) and the empty water pump (15) on the internal circulation pipeline; a fourth valve (23) is arranged between the air compressor (19) and the sterilizer (18) on the second branch pipe; and a fifth valve (24) is arranged on a pipeline connecting the high-temperature steam sterilization device (3) and the second branch pipe.

5. The biological fermentation reactor based on rotary jet stirring according to claim 1, wherein the fermentation tank (1) comprises a tank body (4) and a tank cap (5); the tank cap (5) is sealed on a top of the tank body (4), and the tank cap (5) is provided with an exhaust pipe (13); an outer wall of the tank body (4) is provided with a heating jacket (14), and a temperature probe (6), a pH probe (7) and a dissolved oxygen probe (8) are installed on the outer wall; a bottom of the tank body (4) is provided with a sampling port (11) and a discharging port (12); the tank cap (5) is further provided with a cooler (9); and the cooler (9) is a U-shaped pipe, which is inserted on the tank cap (5) and is provided with a water inlet and a water outlet, and a serpentine surrounding design is employed on a part of the cooler (9) located in the tank body (4).

6. The biological fermentation reactor based on rotary jet stirring according to claim 3, wherein the aeration module (17) is made of a sintered porous alloy material, with a pore size ranging from 5 μm to 5 nm.

7. The biological fermentation reactor based on rotary jet stirring according to claim 3, wherein the sterilizer (18) is composed of multiple layers of polymer material and ceramic filter membranes with pore sizes less than 0.45 μm and 0.22 μm in series and encapsulated by a stainless steel jacket.

8. The biological fermentation reactor based on rotary jet stirring according to claim 3, wherein a top of the feeding groove (16) is provided with a sterile exhaust port, internally provided with a stirring and blending device is built in for pre-mixing preparation of a culture medium, and is equipped with a sealing gland to always keep a closed and sterile state during fermentation.

9. The biological fermentation reactor based on rotary jet stirring according to claim 5, wherein the temperature probe (6), the pH probe (7) and the dissolved oxygen probe (8) are respectively arranged in a circle at an equal distance of 120 degrees in the tank body, and are arranged in three circles from top to bottom.

10. A method for performing biological aerobic or anaerobic fermentation by using the biological fermentation reactor according to claim 1, comprising the following steps:
(i) sealing the fermentation tank completely, and performing high-temperature steam sterilization on the fermentation tank and the feeding aeration circulation system by the high-temperature steam sterilization device;
(ii) inoculating a seed solution into the feeding aeration circulation system by aseptic inoculation, and then sending the seed solution into the fermentation tank through the internal circulation pipeline;
(iii) continuously conveying a culture medium and a fermentation gas into the fermentation tank through the feeding aeration circulation system, and meanwhile, internally circulating materials in the fermentation tank through the internal circulation pipeline and the rotary jet stirring device, and enhancing mass transfer and efficiency; and
(iv) after the fermentation is completed, collecting a fermentation broth, feeding a cleaning liquid into the fermentation tank through the feeding aeration circulation system, and completing cleaning of the whole set of device, and finally adopting the high-temperature steam sterilization device for sterilization.
